(19) United States
(12) Patent Application Publication
(10) Pub. No.: US 2026/0283671 A1
(43) Pub. Date: Sep. 24, 2026

(54) EXTRACTION DEVICE FOR INTRAMEDULLARY NAILS

(71) Applicant: Biomet Manufacturing, LLC, Warsaw, IN (US)

(72) Inventor: Paul Borries, Columbia City, IN (US)

(21) Appl. No.: 19/380,688

(22) Filed: Nov. 5, 2025

Related U.S. Application Data
(60) Provisional application No. 63/743,035, filed on Jan. 8, 2025.

(51) Int. Cl. A61B 17/92; A61B 17/00

(52) U.S. Cl. CPC A61B 17/921; A61B 2017/00477; A61B 2017/924

(57) ABSTRACT

An extraction device for an intramedullary nail can include an extraction bolt, the extraction bolt including a self-tapping threaded end including threads configured to cut new threads into an internal bore of an intramedullary nail, the self-tapping threaded end including a cylindrical shape conforming to a shape of the internal bore.

### CLAIM OF PRIORITY

This application claims the benefit of U.S. Provisional Patent Application Ser. No. 63/743,035, filed on Jan. 8, 2025, the benefit of priority of which is claimed hereby, and which is incorporated by reference herein in its entirety.

### TECHNICAL FIELD

The present application relates to a surgical system and specifically to the extraction of an intramedullary nail.

### BACKGROUND

An intramedullary nail is a rod sized and shaped for surgical implantation into a fractured bone to provide internal fixation and support during the healing process. For example, in a fracture fixation surgery, an intramedullary nail can be inserted into an intramedullary canal of a fractured bone, and a plurality of bone screws can be driven through the fractured bone, and any displaced bone fragments thereof, into the intramedullary nail, such as to help reconstruct natural anatomical features of the fractured bone and prevent movement between the fractured bone and the intramedullary nail.

Intramedullary nails typically have threaded cannulations for attaching insertion or extraction devices. If a nail needs to be removed, but the threads have become damaged, the nail can be very difficult to remove.

### OVERVIEW

The proposed system solves the problem of attaching an extraction bolt to the intramedullary nail with or without the presence of usable threads. To further illustrate apparatuses and methods disclosed herein, a non-limiting list of examples is provided here:

Example 1 can include an extraction device for an intramedullary nail including an extraction bolt, the extraction bolt including a self-tapping threaded end including threads configured to cut new threads into an internal bore of an intramedullary nail, the self-tapping threaded end including a cylindrical shape conforming to a shape of the internal bore.

Example 2 is the apparatus of Example 1, wherein optionally when the extraction bolt is screwed into the intramedullary nail, the extraction bolt is mated to the internal bore of the intramedullary nail along a majority of a length of the threads of the extraction bolt.

Example 3 is the apparatus of any of Examples 1-2, wherein optionally the cylindrical shape of the self-tapping threaded end defines a round, circular cylindrical shape.

Example 4 is the apparatus of any of Example 1-3, wherein optionally opposite side surfaces of the self-tapping threaded end are parallel to each other.

Example 5 is the apparatus of any of Examples 1-4, wherein optionally a minor diameter of the threads and a major diameter of the threads are constant along a length of the self-tapping threaded end and parallel to an elongate axis of the extraction bolt after a tapered lead-end of the extraction bolt.

Example 6 is the apparatus of any of Examples 1-5, wherein optionally the self-tapping threaded end includes at least two cutting flutes extending longitudinally along the extraction bolt and arranged equidistant from each other.

Example 7 is the apparatus of any of Examples 1-6, wherein optionally the extraction bolt is formed of a stainless steel material.

Example 8 is the apparatus of any of Examples 1-7, wherein optionally an end of the extraction bolt opposite the self-tapping threaded end includes a connection portion configured to attach to an extraction rod.

Example 9 is the apparatus of any of Examples 1-8, wherein optionally the extraction rod includes an impaction head configured for use with an impaction tool.

Example 10 can include an extraction bolt including an elongate body extending from a proximal end to a distal end, the distal end including a self-tapping threaded end including threads configured to cut new threads into an internal bore of an intramedullary nail, the self-tapping threaded end including a round, cylindrical shape conforming to a shape of the internal bore, and the proximal end including a connection portion configured to attach to an extraction rod.

Example 11 is the system of Example 10, wherein optionally when the extraction bolt is screwed into the intramedullary nail, the extraction bolt is mated to the internal bore of the intramedullary nail along a majority of a length of the threads of the extraction bolt.

Example 12 is the system of any of Examples 10-11, wherein optionally opposite side surfaces of the self-tapping threaded end are parallel to each other.

Example 13 is the system of any of Examples 10-12, wherein optionally a minor diameter of the threads and a major diameter of the threads are constant along a length of the self-tapping threaded end and parallel to an elongate axis of the extraction bolt after a tapered lead-end of the extraction bolt.

Example 14 is the system of any of Examples 10-13, wherein optionally the self-tapping threaded end includes at least two cutting flutes extending longitudinally along the extraction bolt and arranged equidistant from each other.

Example 15 is the system of any of Examples 10-14, wherein optionally the extraction bolt is formed of a stainless steel material.

Example 16 can include a method of extracting an intramedullary nail including screwing an extraction bolt into an internal bore of an intramedullary nail, wherein the extraction bolt includes a self-tapping threaded end including threads configured to cut new threads into the internal bore of the intramedullary nail, and wherein the self-tapping threaded end includes a cylindrical shape conforming to a shape of the internal bore, and extracting the intramedullary nail.

Example 17 is the method of Example 16, wherein optionally when the extraction bolt is screwed into the intramedullary nail, the extraction bolt is mated to the internal bore of the intramedullary nail along a majority of a length of the threads of the extraction bolt.

Example 18 is the apparatus of any of Examples 16-17, wherein optionally the cylindrical shape of the self-tapping threaded end defines a round, circular cylindrical shape, wherein opposite side surfaces of the self-tapping threaded end are parallel to each other.

Example 19 is the apparatus of any of Examples 16-18, wherein optionally a minor diameter of the threads and a major diameter of the threads are constant along a length of the self-tapping threaded end and parallel to an elongate axis of the extraction bolt after a tapered lead-end of the extraction bolt.

Example 20 is the apparatus of any of Examples 16-19, wherein optionally the extraction bolt is attached to an extraction rod, and wherein the extraction rod includes an impaction head configured for use with an impaction tool.

In Example 21, the apparatuses or methods of any one or any combination of Examples 1-20 can optionally be configured such that all elements or combinations of elements or options recited are available to use or select from.

These and other examples and features of the present apparatuses and methods will be set forth in part in the following Detailed Description. This Overview is intended to provide non-limiting examples of the present subject matter—it is not intended to provide an exclusive or exhaustive explanation. The Detailed Description below is included to provide further information about the present apparatus, systems, and methods.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 shows a cross section view of intramedullary nail within a femur, in accordance with one embodiment.

FIG. 2 shows a side view of an extraction bolt, in accordance with one embodiment.

FIG. 3 shows a cross-section view of the extraction bolt, in accordance with one embodiment.

FIG. 4 shows a close up side view of a self-tapping threaded end of the extraction bolt, in accordance with one embodiment.

FIG. 5 shows an end view of the self-tapping threads, in accordance with one embodiment.

FIG. 6 shows a side view of the extraction bolt before insertion of the extraction bolt into the intramedullary nail, in accordance with one embodiment.

FIG. 7 shows a side view of the extraction bolt inserted into the intramedullary nail, in accordance with one embodiment.

FIG. 8 shows a side view of an extraction rod coupled to the extraction bolt, in accordance with one embodiment.

FIG. 9 shows a method of extracting an intramedullary nail, in accordance with one embodiment.

### DETAILED DESCRIPTION

As used herein, the following directional definitions apply. Anterior and posterior mean nearer the front or nearer the rear of the body, respectively, proximal and distal mean nearer to or further from the root of a structure, respectively, and medial and lateral mean nearer the sagittal plane or further from the sagittal plane, respectively. The sagittal plane is an imaginary vertical plane through the middle of the body that divides the body into right and left halves.

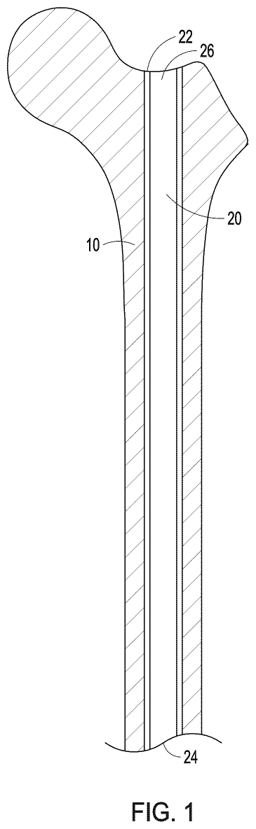

FIG. 1 shows a cross section view of an intramedullary nail 20 within a femur 10, showing the context of the present extraction system. Here, the nail 20 includes an elongate main body extending from a proximal end 22 to a distal end 24 configured to be inserted into a medullary canal of a bone 10. The elongate main body of the nail 20 can include a solid outer surface and a hollow inner bore 26 (i.e. a cannulation). The proximal end 22 of the elongate body optionally includes internal threads within the bore 26. Some intramedullary nails can include external threads. The threads are used to attach to a nail insertion guide when implanting the nail 20.

As noted, in some situations intramedullary nails may need to be removed, but the internal threads may become damaged, and it can be very difficult to remove the nail. Presently, some intramedullary nail extractors have conical threads which are designed to attach to a variety of nail designs and diameter sizes. However, these conical extractors are inadequate at attaching to the proximal end of the nail. Specifically, the conical extractors do not attach very well to the nail and disconnect when impacted since the connection at the threads between the conical threads and the internal bore of the nail does not extend for very long along the internal bore of the nail.

As will be detailed below, the proposed extraction system solves the problem of attaching to the nail cannulation with or without the presence of usable threads. As will be discussed, the present system creates a new threaded attachment between the extractor and the intramedullary nail with a solid connection that can then be used for extraction.

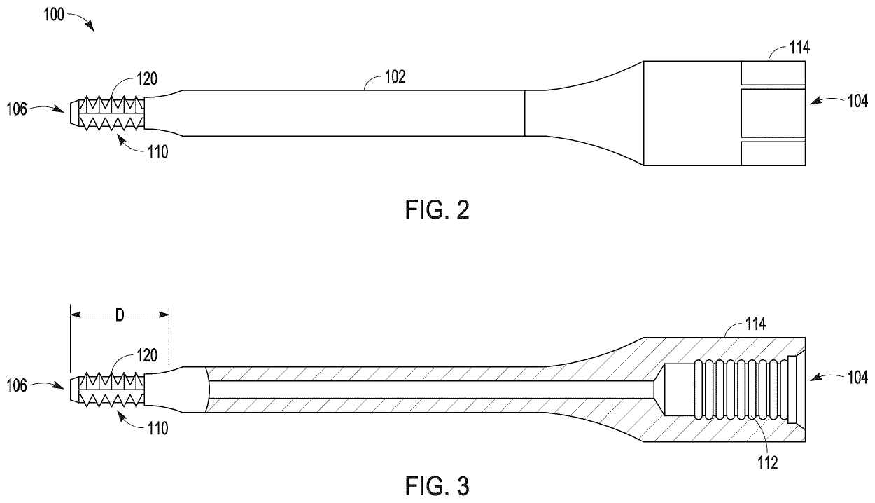

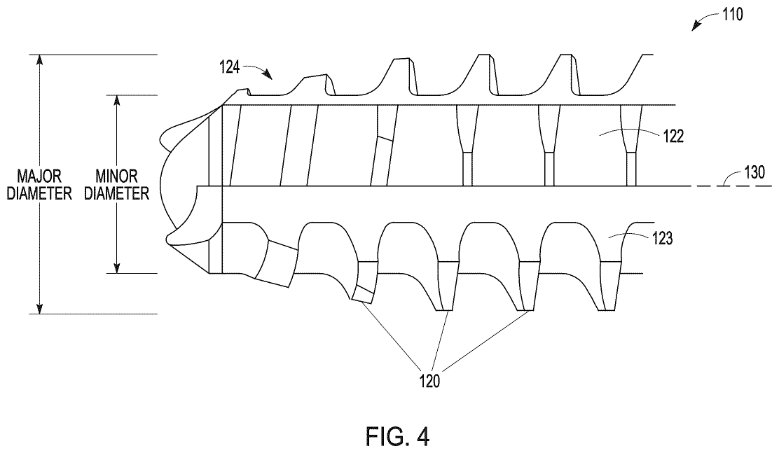

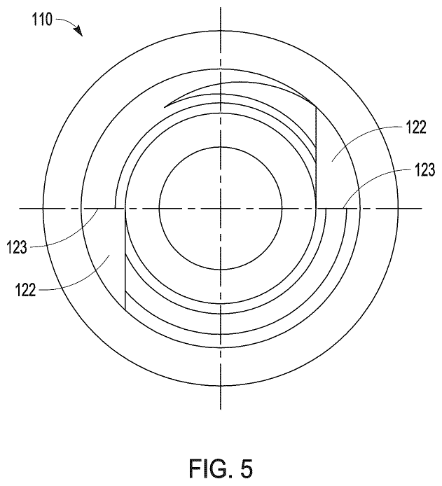

FIG. 2 shows a side view of an extraction bolt 100, in accordance with one embodiment, FIG. 3 shows a cross-section view of the extraction bolt, in accordance with one embodiment, FIG. 4 shows a close up side view of a self-tapping threaded end of the extraction bolt, in accordance with one embodiment, and FIG. 5 shows an end view of the self-tapping threads, in accordance with one embodiment, in accordance with one embodiment.

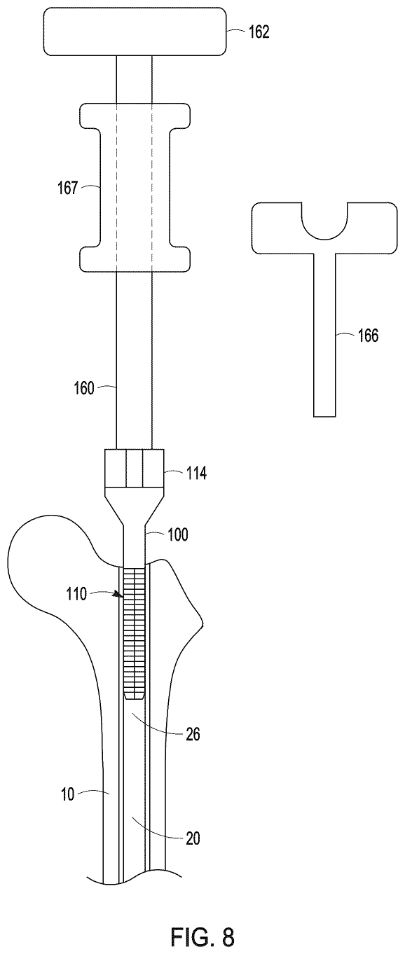

In this example, the extraction bolt 100 includes an elongate body 102 extending from a proximal end 104 to a distal end 106. The distal end 106 can include a self-tapping threaded end 110 including cutting threads 120 configured to cut new threads into the internal bore 26 of the intramedullary nail 20 (FIG. 1). The self-tapping threaded end 110 can include a round, cylindrical shape conforming to a cylindrical shape of the internal bore 26. The extraction bolt can further include a hex feature 114 or other tightening mechanism to be used to rotate the extraction bolt 100 so as to screw the extraction bolt 100 into the internal bore of the nail. The proximal end 104 can include a connection portion, such as threads 112 configured to attach to an extraction rod 160 (FIG. 8). In some examples, the extraction bolt 100 can be used on its own without an extraction rod, and the proximal end 104 can include a structure to interact with a slap-hammer or slotted mallet, for example.

In one example, for instance, for a 5/16″ ID intramedullary nail, the self-tapping threaded end 110 can use 5/16″-24 self-tapping threads. For other nails having other assorted inner diameters, an appropriately sized extraction bolt can be used.

By using an extraction bolt having a round cylindrical shape (as opposed to a conical shape), when the extraction bolt 100 is screwed into the intramedullary nail 20, the extraction bolt 100 is mated to the internal bore 26 of the nail 20 along a majority of a length of the threads 120 of the extraction bolt 100. For example, 50%-100% of the threads 120 are cut into and mated to the internal bore 26. This provides for thread purchase along the entire length of the extraction bolt 100 bolt with a majority of the threads providing gripping force. Again, this is in contrast to a conical bolt, which typically provides a one or two thread purchase, and thus is prone to disconnect when impacted.

As noted, the cylindrical shape of the self-tapping threaded end 110 defines a round, circular cylindrical shape. Thus, opposite side surfaces of the self-tapping threaded end 110 are parallel to each other. In this example, as shown in FIG. 4, after a tapered lead-end portion 124 of the extraction bolt 100, a minor diameter of the threads 120 and a major diameter of the threads 120 are constant along a length of the self-tapping threaded end and parallel to each other and to an elongate axis 130 of the extraction bolt 100. In some examples, the minor diameter of the threaded end 110 is always constant along the entire length of the threaded end 110 and only the major diameter is smaller for one to three threads during the tapered lead-end portion 124. Again, after the lead-end portion 124, the major diameter is also constant along the rest of the length of the threaded end 110.

In one example, the self-tapping threaded end 110 includes two or more cutting flutes 122 extending longitudinally along the extraction bolt 10 and arranged equidistant from each other. In one example, the flutes 122 can extend along the threaded end 110 and into a non-threaded portion of the bolt a distance D, as shown in FIG. 3. In some embodiments, the threaded end can include three, four or more cutting flutes 122 located equidistantly around the circumference of the bolt. The cutting flutes 122 can include a cutting edge 123 that cuts into the material of the inner bore of the intramedullary nail to cut and define new threads.

In an example, the extraction bolt 100 can be formed of a stainless steel material or another material that is harder than the titanium or aluminum alloys typically used in intramedullary nails so as to displace the metal of the nail 20, and thus cut fresh new threads into the inner bore of the nail.

Accordingly, this extraction system is configured to cut new threads into the cannulated nail or restore damaged threads by using a self-tapping thread design. The extractor can have several different size options. Once the tap end is threaded into the nail, it becomes rigidly fixed by creating new threads and remains threaded into the new or repaired threads. A slap-hammer or extraction bolt is then connected to the opposite end of the extraction bolt and the whole construct can be impacted back out of the intramedullary canal.

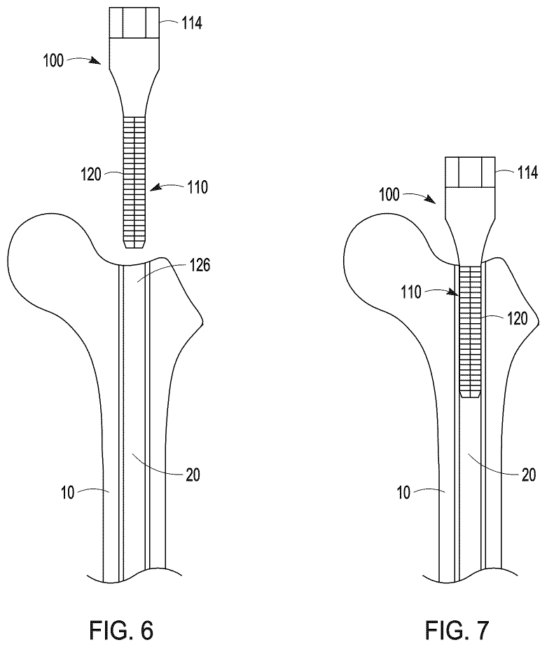

FIG. 6 shows a side view of the extraction bolt 100 before insertion of the extraction bolt 100 into the intramedullary nail 20, in accordance with one embodiment, and FIG. 7 shows a side view of the extraction bolt 100 inserted into the intramedullary nail 20, in accordance with one embodiment.

In use, the user can use the hex feature 114 or other means to screw the bolt 100 into the proximal end of the inner bore 26 of the nail 20. As the extraction bolt 100 is screwed into the bore 26, new threads are cut along the walls of the bore. As shown in FIG. 7, this results in the threads 120 mating with the walls of the inner bore 26 along a majority (or the entire) length of the threaded end 110. Again, this provides for thread purchase along the length of the end 110 of the bolt 100 with a majority of the threads 120 providing gripping force. This is opposed to a conical bolt which typically provides one or two thread purchase.

FIG. 8 shows a side view of an extraction rod 160 coupled to the extraction bolt 100, in accordance with one embodiment.

Here, once the bolt 100 is installed into the inner bore 26 of the nail 20, the extraction rod 160 can be attached to the proximal end of the extraction bolt 100, using threads 112, for example. (See FIG. 2). In this example, the extraction rod 160 includes an impaction head 162 configured for use with an impaction tool, such as a slap-hammer 164 or a slotted mallet 166, for example. The impaction tool can be used to impact against the impaction head 162 of the extraction rod to back the nail out of the intramedullary canal. As noted above, in some examples the extraction bolt 100 can be used on its own without an extraction rod, and the proximal end of the extraction blot 100 can include a structure to interact with the slap-hammer or the slotted mallet, for example.

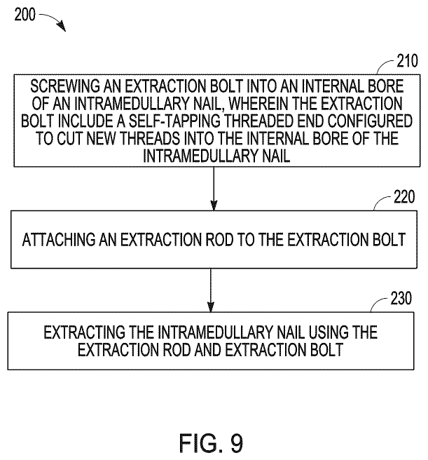

FIG. 9 shows a method (200) of extracting an intramedullary nail, in accordance with one embodiment.

Referring to the Figures above, the method (200) can include screwing an extraction bolt into an internal bore of an intramedullary nail (210). The extraction bolt can include a self-tapping threaded end configured to cut new threads into the internal bore of the intramedullary nail. The self-tapping threaded end can include a cylindrical shape conforming to the cylindrical shape of the internal bore. In one option, the method can further include attaching an extraction rod to the extraction bolt (220) and extracting the intramedullary nail (230) by using a slap-hammer or other impaction device and impacting against an impaction head of the extraction rod to back nail out of the intramedullary canal. In another option, the extraction bolt 100 can be used on its own without an extraction rod.

In various embodiments, the method can include the extraction bolt being mated to the internal bore of the nail along a majority of a length of the threads of the extraction bolt. The cylindrical shape of the self-tapping threaded end of the extraction bolt can define a round, circular cylindrical shape, such that opposite side surfaces of the self-tapping threaded end are parallel to each other. Moreover, after a tapered lead-end of the extraction bolt, the minor diameter of the threads and the major diameter of the threads are constant along a length of the self-tapping threaded end and parallel to an elongate axis of the extraction bolt.

In summary, in some situations intramedullary nails may need to be removed, but the threads have become damaged and it can be very difficult to remove the nail.

The present extraction system solves the problem of attaching to the nail cannulation with or without the presence of usable threads. For example, the present system can create a new threaded attachment between the extractor bolt and the nail with a solid connection that can then be used for extraction.

This extraction system is configured to cut new threads into the nail or restore damaged threads by using a thread tap design. The extractor can have several different size options. The end that attaches to the nail can have a self-tapping design, and the opposite end will have standard threads for attaching to a slap-hammer or extraction bolt. Once the self-tapping end is threaded into the nail, it becomes rigidly fixed by creating new threads and remaining threaded into the new or repaired threads. The slap-hammer or extraction bolt is then connected to the opposite end of the instrument and the whole construct can be impacted back out of the intramedullary canal.

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls. In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. § 1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

The claimed invention is:

1. An extraction device for an intramedullary nail, the extraction device comprising:
an extraction bolt, the extraction bolt including a self-tapping threaded end including threads configured to cut new threads into an internal bore of an intramedullary nail, the self-tapping threaded end including a cylindrical shape conforming to a shape of the internal bore.

2. The extraction device of claim 1, wherein when the extraction bolt is screwed into the intramedullary nail, the extraction bolt is mated to the internal bore of the intramedullary nail along a majority of a length of the threads of the extraction bolt.

3. The extraction device of claim 1, wherein the cylindrical shape of the self-tapping threaded end defines a round, circular cylindrical shape.

4. The extraction device of claim 3, wherein opposite side surfaces of the self-tapping threaded end are parallel to each other.

5. The extraction device of claim 4, wherein a minor diameter of the threads and a major diameter of the threads are constant along a length of the self-tapping threaded end and parallel to an elongate axis of the extraction bolt after a tapered lead-end of the extraction bolt.

6. The extraction device of claim 1, wherein the self-tapping threaded end includes at least two flutes extending longitudinally along the extraction bolt and arranged equidistant from each other.

7. The extraction device of claim 1, wherein the extraction bolt is formed of a stainless steel material.

8. The extraction device of claim 1, wherein an end of the extraction bolt opposite the self-tapping threaded end includes a connection portion configured to attach to an extraction rod.

9. The extraction device of claim 8, wherein the extraction rod includes an impaction head configured for use with an impaction tool.

10. An extraction bolt comprising:
an elongate body extending from a proximal end to a distal end;
the distal end including a self-tapping threaded end including threads configured to cut new threads into an internal bore of an intramedullary nail, the self-tapping threaded end including a round, cylindrical shape conforming to a shape of the internal bore; and
the proximal end including a connection portion configured to attach to an extraction rod.

11. The extraction bolt of claim 10, wherein when the extraction bolt is screwed into the intramedullary nail, the extraction bolt is mated to the internal bore of the intramedullary nail along a majority of a length of the threads of the extraction bolt.

12. The extraction bolt of claim 10, wherein opposite side surfaces of the self-tapping threaded end are parallel to each other.

13. The extraction bolt of claim 12, wherein a minor diameter of the threads and a major diameter of the threads are constant along a length of the self-tapping threaded end and parallel to an elongate axis of the extraction bolt after a tapered lead-end of the extraction bolt.

14. The extraction bolt of claim 13, wherein the self-tapping threaded end includes at least two flutes extending longitudinally along the extraction bolt and arranged equidistant from each other.

15. The extraction bolt of claim 10, wherein the extraction bolt is formed of a stainless steel material.

16. A method of extracting an intramedullary nail, the method comprising:
screwing an extraction bolt into an internal bore of an intramedullary nail, wherein the extraction bolt includes a self-tapping threaded end including threads configured to cut new threads into the internal bore of the intramedullary nail, and wherein the self-tapping threaded end includes a cylindrical shape conforming to a shape of the internal bore; and
extracting the intramedullary nail.

17. The method of claim 16, wherein when the extraction bolt is screwed into the intramedullary nail, the extraction bolt is mated to the internal bore of the intramedullary nail along a majority of a length of the threads of the extraction bolt.

18. The method of claim 16, wherein the cylindrical shape of the self-tapping threaded end defines a round, circular cylindrical shape, wherein opposite side surfaces of the self-tapping threaded end are parallel to each other.

19. The method of claim 18, wherein a minor diameter of the threads and a major diameter of the threads are constant along a length of the self-tapping threaded end and parallel to an elongate axis of the extraction bolt after a tapered lead-end of the extraction bolt.

20. The method of claim 16, wherein the extraction bolt is attached to an extraction rod, and wherein the extraction rod includes an impaction head configured for use with an impaction tool.
